(19) United States
(12) Patent Application Publication
(10) Pub. No.: US 2007/0116686 A1
(43) Pub. Date: May 24, 2007

(54) Method for separating precursor cells producing gabaergic neuron alone

(76) Inventor: Nobuaki Tamamaki, Kumamoto (JP)

Correspondence Address: WENDEROTH, LIND & PONACK, L.L.P., 2033 K STREET N. W., SUITE 800, WASHINGTON, DC, 20006-1021, US

(21) Appl. No.: 10/539,349

(22) PCT Filed: Dec. 17, 2003
(86) PCT No.: PCT/JP03/16188

§ 371 Date: Oct. 20, 2005

(30) Foreign Application Priority Data
Dec. 18, 2002 (JP) 2002-367272
Feb. 20, 2003 (JP) 2003-42253

(51) Int. Cl. A61K 48/00; C12N 5/08

(52) U.S. Cl. 424/93.21; 424/93.7; 514/44; 435/368

(57) ABSTRACT

To treat epilepsy or schizophrenia by transplanting precursor cells of GABAergic neurons into a region where GABAergic neurons are lost or decreased in the brain of a patient suffering from the disease, it is intended to provide a method for separating a precursor cell of GABAergic neuron in an adult or a fetal nerve tissue or a precursor cell of GABAergic neuron derived from an embryo stem cell. The invention of this application comprises the step of preparing a cell population containing a precursor cell of GABAergic neuron, the step of introducing a DNA, in which a reporter gene emitting fluorescence detectable even in vivo is attached to the downstream of a promoter of GAD67 gene or GAD65 gene that is gene of an inhibitory neurotransmitter GABA synthase, into dispersed cells, the step of isolating a GABAergic neuron and a precursor cell of GABAergic neuron based on the presence/absence of the fluorescence from the reporter protein, and the step of isolating the precursor cell of GABAergic neuron based on the possession of proliferative capability.

### TECHNICAL FIELD

The invention of this application relates to a method for separating a precursor cell of GABAergic neuron that produces a GABAergic neuron alone, which is used for restoring the number of inhibitory neurons in a region where inhibitory neurons are lost or decreased to a normal level. More particularly, the invention of this application relates to a method for separating a precursor cell as a medical material or the like enabling a medical treatment in which epilepsy or schizophrenia is treated by restoring a region where GABAergic neurons are lost or decreased to normal by enabling the separation of a precursor cell of GABAergic neuron.

### BACKGROUND ART

As for neurons in the central nervous system, there are excitatory neurons and inhibitory neurons. Both neurons are contained in a variety of different ratios depending on regions of the central nerve, and information processing is carried out. In the cerebral cortex, inhibitory neurons use γ-aminobutyric acid (GABA) as a neurotransmitter, and excitatory neurons use glutamate. The inhibitory neurons in the cerebral cortex are present at a ratio of about 20% of neurons, whereby an appropriate activity level can be maintained in the whole neural circuit, and information processing can be carried out smoothly. In some cases, however, all the neurons begin to be excited, which results in the occurrence of an epileptic seizure in which consciousness is lost. Although the causes of inducing such a seizure include the febrile convulsion, which is caused because the development of the neural circuit of the brain is immature during the childhood, therefore a neuron is easy to be excited by the onset of fever, many cases have genetic background. Many of the epileptic patients have a point mutation in a channel molecule that is involved in the excitement of neurons, therefore it is considered that they are easy to be excited. On the other hand, in the case where the molecular mechanism of cell migration is abnormal, the gray matter of the cerebral cortex is dichotomized, input and output relation becomes unbalanced, and an epileptic-like seizure may be repeated in some cases. All the cases are a short circuit like abnormal condition occurring in the neural circuit, and it is considered that a flow of a large amount of calcium into a cell body due to over-ignition results in cell death. However, this does not occur in all neurons; inhibitory neurons whose role is to suppress the occurrence of such a short circuit like situation particularly receive excess input and they die faster than other excitatory neurons. Such a condition is referred to as a focus of an intractable epileptic seizure and inhibitory neurons in the focus dramatically decrease and the focus becomes an origin of the occurrence of repeated epileptic seizures. Intractable epilepsy in a condition like this cannot be fully treated only with a therapeutic drug, and a radical therapy, in which the focus region is excised to suppress the occurrence of an epileptic seizure, is conducted. However, because part of the brain is excised, a function possessed by the excised brain is lost. If precursor cells of GABAergic neurons can be supplied by transplantation to such a focus of an epileptic seizure and they can be allowed to survive, an epileptic seizure can be expected to be suppressed. A GABAergic neuron that is necessary for this purpose may not be any type of GABAergic neuron, but it must be a certain type of GABAergic neuron that can suppress the activity of excitatory neuron in the cerebral cortex among over one hundred of subtypes of GABAergic neurons. For example, with regard to which subtype of GABAergic neuron needs to be transplanted to the focus of an epileptic patient, a basket cell that gives suppression to part of a cell body or a chandelier cell that gives suppression to an axon hillock, which can resist and suppress excitatory input that is sent back from surrounding excitatory neurons for every time when the excitatory neurons are excited, is necessary.

The origin of GABAergic neuron in the human cerebral neocortex has not been fully understood up to the present day. The inventor of this application found that the origin of GABAergic neuron in the rodent cerebral cortex originates in the ganglionic eminence so far and made a report on Nov. 1, 1997 (Tamamaki et al., J. Neurosci. 17: 8313-8323, 1997). In addition, Anderson S. in the US also made a similar report on Oct. 27, 1997 (Anderson et al., Science 278: 474-476, 1997) other than this. Further, it has been reported that the origin thereof is limited to the medial ganglionic eminence among the ganglionic eminence (Lavdas et al., J. Neurosci. 19: 7881-7888, 1999). The fact that it is limited to the medial ganglionic eminence among the ganglionic eminence has been also confirmed by the transplantation of a fetal tissue (Wichterle et al., Development 128: 3759-3771, 2001). However, it has not been confirmed whether or not there is an origin other than the ganglionic eminence. In such a circumstance, there is a report that the origin of GABAergic neuron is also in the cerebral cortex in human, and 65% thereof is produced in the cerebral cortex and 35% thereof is produced in the ganglionic eminence (Letinic et al., Nature 417: 645-649, 2002). It was considered that 65% of the precursor cells of GABAergic neurons derived from the cerebral cortex were supplied by the division of the neural stem cells present in the ventricular zone to the subventricular zone and were characterized by Mash1 positive. Such observation results partially agree with the individual observation results of the latest research in which the origin of GABAergic neuron in a rodent was studied by the inventor of this application, however, the interpretation of the origin is very different from that of the inventor. According to the study using rodents by the inventor of this application, it is considered that the origin of GABAergic neuron is in the ganglionic eminence, and part of GABAergic neurons that migrate to the cerebral cortex dedifferentiate into precursor cells, or part of GABA-containing cells that migrate to the cerebral cortex are precursor cells, and GABAergic neurons are newly supplied in the cerebral cortex. In the case of human, in view of the agreement of the observation results, it is considered that GABAergic neurons in the cerebral cortex are derived from cells in the ganglionic eminence.

However, precursor cells of GABAergic neurons are not only found in the cerebral neocortex. If an appropriate culture condition is provided when ES cells or neural stem cells are cultured, the cells begin to differentiate into neural precursor cells, and many of the differentiated neural precursor cells begin to produce also GABAergic neurons. If these precursor cells of GABAergic neurons can be supplied by transplantation to the seizure focus of an epileptic patient and they can be allowed to survive, an epileptic seizure can be expected to be suppressed. However, GABAergic neurons obtained under a culture condition can be obtained only as a mixture of neurons, which do not contain GABA, and glial cells so far.

Incidentally, as the publications associated with the invention of this application, there are the following publications including the ones already cited.

### PUBLICATION LIST

1. Anderson S A, Eisenstat D D, Shi L, Rubenstein J L R. (1997) Interneuron migration from the basal forebrain to the neocortex: dependence on Dlx genes. Science 278: 474-476.

2. Dupuy S T, Houser C R. (1996) Prominent expression of two forms of glutamate decarboxylase in the embryonic and early postnatal rat hippocampal formation. J Neurosci 16:6919-6932.

3. Gritti A, Parati E A, Cova L, Frolichsthal P, Galli R, Wanke E, Faravelli L, Morassutti D J, Roisen F, Nickel D D, Vescovi A L. (1996) Multipotential stem cells from the adult mouse brain proliferate and self-renew in response to basic fibroblast growth factor. J Neurosci 16: 1091-1100.

4. Jin, X, Mathers P H, Szabo G, Katarova Z, Agmon A. (2001) Vertical bias in dendritic trees of non-pyramidal neocortical neurons expressing GAD67-GFP in vitro. Cereb Cortex, 11, 666-678.

5. Lavdas A A, Grigoriou M, Pachnis V, Parnavelas J G. (1999) The medial ganglionic eminence gives rise to a population of early neurons in the developing cerebral cortex. J Neurosci 19: 7881-7888.

6. Letinic K, Zoncu, R, Rakic P. (2002) Origin of GABAergic neurons in the human neocortex. Nature, 417: 645-649.

7. Nakamura K, Nakamura K, Kometani K, Yanagawa Y, Iwasato T, Obata K, Minato K, Kaneko T, Tamamaki N. (2003) Immigration of the proliferative progenitors for GABAergic neurons from the ganglionic eminence to the neocortex. Society for Neurosci. Abst. 33th.

8. Porteus M H, Bulfone A, Liu J K, Puelles L, Lo L C, Rubenstein J L. (1994) DLX-2, MASH-1, and MAP-2 expression and bromodeoxyuridine incorporation define molecularly distinct cell populations in the embryonic mouse forebrain. J Neurosci 14: 6370-6383.

9. Reynolds B A, Weiss S. (1992) Generation of neurons and astrocytes from isolated cells of the adult mammalian central nervous system. Science 255: 1707-10.

10. Tamamaki N, Fujimori K, Takauji R. (1997) Origin and route of tangentially migrating neurons in the developing neocortical intermediate zone. J. Neurosci. 17: 8313-8323.

11. Tamamaki N, Sugimoto Y, Tanaka K, Takauji R. (1999) Cell migration from the ganglionic eminence to the neocortex investigated by labeling nuclei with UV irradiation via a fiber optic cable. Neurosci Res. 35: 241-251.

12. Tamamaki N, Yanagawa Y, Tomioka R, Miyazaki J, Obata K, Kaneko T. (2003) Green fluorescent protein expression and colocalization with calretinin, parbalbumin, and somatostatin in the gad67-gfp knock-in mouse. J Comp Neurol 467: 60-79.

13. Vescovi A L, Reynolds B A, Fraser D D, Weiss S. (1993) bFGF regulates the proliferative fate of unipotent (neuronal) and bipotent (neuronal/astroglial) EGF-generated CNS progenitor cells. Neuron 11: 951-966.

14. Westmoreland J J, Hancock C R, Condie B G (2001) Neuronal development of embryonic stem cells: a model of GABAergic neuron differentiation. Biochem Biophys Res Commun 284: 674-680.

### DISCLOSURE OF THE INVENTION

As an invention to solve the above-mentioned problems, this application provides a method for separating a precursor cell producing a GABAergic neuron alone, which comprises the steps of:

(a) preparing a cell population containing a precursor cell of GABAergic neuron;

(b) introducing a DNA, in which a cDNA of a reporter protein emitting a signal detectable even in vivo is attached to the downstream of a promoter of GAD67 gene or GAD65 gene that is gene of an inhibitory neurotransmitter GABA synthase, into each cell in the cell population;

(c) isolating a GABAergic neuron and a precursor cell of GABAergic neuron based on the presence/absence of the signal emitted by the reporter; and

(d) isolating the precursor cell of GABAergic neuron based on the possession of proliferative capability.

In addition, the invention of this application provides a method for separating a precursor cell producing a GABAergic neuron alone, which comprises the steps of:

(a) preparing a cell population containing a precursor cell of GABAergic neuron;

(b) introducing a DNA, in which a CDNA of a protein imparting a property of drug resistance is attached to the downstream of a promoter of GAD67 gene or GAD65 gene that is gene of an inhibitory neurotransmitter GABA synthase, into each cell in the cell population;

(c) isolating a GABAergic neuron and a precursor cell of GABAergic neuron based on the presence/absence of the drug resistance; and

(d) isolating the precursor cell of GABAergic neuron based on the possession of proliferative capability.

Further, the invention of this application provides a method for separating a precursor cell producing a GABAergic neuron alone, which comprises the steps of:

(a) preparing a cell population containing a precursor cell of GABAergic neuron;

(b) introducing a DNA, in which a cDNA of a recombinant enzyme and a cassette DNA are attached to the downstream of a promoter of GAD67 gene or GAD65 gene that is a gene of an inhibitory neurotransmitter GABA synthase, into each cell in the cell population, wherein the cassette DNA expresses a reporter protein emitting a signal detectable even in vivo after being genetically recombined;

(c) isolating a GABAergic neuron and a precursor cell of GABAergic neuron based on the presence/absence of the signal emitted by the reporter; and

(d) isolating the precursor cell of GABAergic neuron based on the possession of proliferative capability.

Still further, the invention of this application provides a method for separating a precursor cell producing a GABAergic neuron alone, which comprises the steps of:

(a) preparing a cell population containing a precursor cell of GABAergic neuron;

(b) introducing a DNA, in which a cDNA of a recombinant enzyme and a cassette DNA are attached to the downstream of a promoter of GAD67 gene or GAD65 gene that is gene of an inhibitory neurotransmitter GABA synthase, into each cell in the cell population, wherein the cassette DNA expresses a protein imparting a property of drug resistance after being genetically recombined;

(c) isolating a GABAergic neuron and a precursor cell of GABAergic neuron based on the presence/absence of the drug resistance; and

(d) isolating the precursor cell of GABAergic neuron based on the possession of proliferative capability.

In each of the above-mentioned inventions, it is preferred to carry out the steps in the order of (a) to (d), however, it is not limited thereto, and the order of the respective steps can be changed as needed. For example, it may be in the order of (a), (d), (b) and (c), or (a), (b), (d) and (c). In addition, the step (b) can be carried out by, for example, producing a transgenic animal with the use of an expression cassette DNA as an introduced gene. In this case, each invention can be carried out in the order of, for example, (b), (a), (c) and (d), or (b), (a), (d) and (c).

In addition, with regard to each of the above-mentioned inventions, in the step (a), a preferred embodiment is that a cell population contains a precursor cell of GABAergic neuron induced from an embryo stem cell or a neural stem cell, or that a cell population is prepared by dispersing tissues containing a precursor cell of GABAergic neuron of a donor.

Further, in each of the above-mentioned inventions, a preferred embodiment is that the method of introducing a DNA includes any one of transformation mediated by a virus, electroporation and transformation mediated by a liposome.

In addition, in each of the above-mentioned inventions, another preferred embodiment is that the donor is a mammal, and the mammal is human.

Still further, in each of the above-mentioned inventions, a preferred embodiment is that a step of transplanting the cell separated in the step (d) into a recipient is further included.

Further, the invention of this application provides a precursor cell producing a GABAergic neuron alone obtained by a method according to any one of the above-mentioned inventions.

Still further, the invention of this application provides a kit containing a reagent and a cell, which is used for obtaining a precursor cell producing a GABAergic neuron alone in a method according to any one of the above-mentioned inventions.

Incidentally, in the following description, “promoter of GAD67 gene” is referred to as “GAD67 promoter”, and “promoter of GAD65 gene” is referred to as “GAD65 promoter” in some cases.

### BRIEF DESCRIPTION OF DRAWINGS

FIG. 1 shows DNA constructs 1 to 5, which are necessary for carrying out the method of this invention. The DNA constructs 1 and 2 are the one in which a GFP gene or a neomycin-resistance gene is directly attached to GAD67 promoter, and GFP or a neomycin-resistance gene is expressed in a GABAergic neuron and a precursor cell of GABAergic neuron, whereby they are utilized to separate a GABAergic neuron and a precursor cell of GABAergic neuron. The DNA constructs 3 to 5 are utilized to separate a precursor cell of GABAergic neuron by expressing GFP or a neomycin-resistance gene in a GABAergic neuron and a precursor cell of GABAergic neuron with the use of Cre recombinase, which is expressed by the activity of GAD67 promoter. To be specific, the DNA construct 3 is a DNA construct in which a Cre recombinase DNA is attached to GAD67 promoter, the DNA construct 4 is a DNA construct to be used in a method of separating a GABAergic neuron and a precursor cell of GABAergic neuron by utilizing the expression of GFP mediated by Cre recombinase, and the DNA construct 5 is a DNA construct to be used in a method of separating a GABAergic neuron and a precursor cell of GABAergic neuron by utilizing the expression of neomycin- resistance gene mediated by Cre recombinase.

FIG. 2 is an explanatory diagram of an embodiment of the invention. FIG. 2-1 shows a method of separating a GABAergic neuron and a precursor cell of GABAergic neuron by utilizing a drug-resistance gene (neomycin-resistance gene). In the case where the two types of DNA constructs (DNA constructs 3 and 5 in FIG. 1) shown in the figure are introduced into a cell, Cre recombinase is expressed in a GABAergic neuron and a precursor cell of GABAergic neuron due to the activity of GAD67 promoter, the stop signal DNA is removed and neomycin resistance is acquired, whereby the cells can be selected with Geneticin or the like due to the expression of a neomycin degradative enzyme. The introduction of a gene can be carried out by using a retrovirus, an adenovirus having a replication origin, which allows temporary expression in a eukaryotic cell, or the like. FIG. 2-2 shows a method of separating a GABAergic neuron and a precursor cell of GABAergic neuron by utilizing a reporter DNA (GFP), which can visualize a cell in a living organism. In the case where the two types of DNA constructs (DNA constructs 3 and 4 in FIG. 1) shown in the figure are introduced into a cell, Cre recombinase is expressed in a GABAergic neuron and a precursor cell of GABAergic neuron due to the activity of GAD67 promoter, the stop codon sequence is removed and the expression of GFP is initiated by CA promoter. As a result, based on the presence/absence of GFP fluorescence, the GABAergic neuron and the precursor cell of GABAergic neuron can be selected by using a cell sorter. The introduction of the above-mentioned gene can be carried out by using a retrovirus, an adenovirus having a replication origin, which allows temporary expression in a eukaryotic cell, or the like.

FIG. 3 shows data when a GFP-positive GABAergic neuron and a GFP-positive precursor cell of GABAergic neuron obtained from a mouse into which a DNA, in which a cDNA of GFP was attached to the downstream of GAD67 promoter, was knocked in were separated using a cell sorter. In the upper left is a graph illustrating the relationship between the number of cells and the fluorescence intensity of GFP. The cells in the range, which significantly emit fluorescence compared with the control, were collected. The collected cells were cultured in a culture medium which had been prepared by culturing the cerebral cortex and the ganglionic eminence for one day in advance with a basal culture medium to which only a cell growth factor was added. Further, BrdU was added, and the DNA synthesis during the cell growth was detected. As shown in the upper right figure, BrdU was incorporated into the precursor cell of GABAergic neuron, however, when a DNA synthesis inhibitor was added, incorporation of BrdU into the DNA was not observed. The lower figure shows images showing that the GFP-positive precursor cell of GABAergic neuron, in which BrdU had been incorporated into the DNA in two nuclei, was undergoing cell division.

### BEST MODE FOR CARRYING OUT THE INVENTION

In the above-mentioned inventions of this application, while a neural stem cell is a cell capable of supplying all types of cells constituting the central nervous system, a “precursor cell” means a cell that is produced from a neural stem cell and can proliferate, but can differentiate into a limited type of cell. For example, as a precursor cell of oligodendrocyte cell, O-2A progenitor cell is known, and as a precursor cell of GABAergic neuron, a precursor cell in the subventricular zone of the forebrain vesicle, which supplies an olfactory bulb granule cell, is known. However, it was not known that a precursor cell, which produces a GABAergic neuron of the cerebral neocortex, is present in the parenchyma of the cerebral neocortex so far. The following observation results by the inventor of this application is a direct and indirect evidence that a precursor cell of GABAergic neuron is present in the brain parenchyma of the cerebral cortex.

1. BrdU was incorporated into an embryonic E16 mouse, and immediately perfusion fixation was carried out. Then, double labeling with BrdU and MAP2 was carried out, whereby a MAP2-positive migrating cell in the intermediate zone, which shows BrdU immunoreactivity, is found. This indicates that the MAP2-positive migrating neuron in the intermediate zone (GABA-containing neuron) replicated DNA in preparation for cell division.

2. Even when a GAD67-GFP knock-in mouse shortly after birth is subjected to a similar BrdU pulse labeling, there are some cells showing BrdU immunoreactivity among the GFP-positive GABAergic neurons, which are confirmed by double labeling. It is indicated that the double-labeled GFP-positive neuron replicated DNA in preparation for cell division.

3. According to the experiment carried out by the inventor, an adenovirus that allows GAP43-EGFP to be expressed is injected into the fetal lateral ventricle of the brain to infect the ventricular zone. On day 20 after birth, GFP-positive neurons are observed. When the virus is injected before E17, lots of non-pyramidal cells that are considered to be GABAergic neurons are observed. However, when the virus is injected after E17, non-pyramidal cells are not observed. On the other hand, according to a report from several other research institutions, in the experiment of injection of BrdU, GABAergic neurons are supposed to continue to divide and proliferate after E14 until birth. These two experiments indicate that a precursor cell that supplies a GABAergic neuron to the cerebral cortex after E17 is not present in the ventricular zone, which an adenovirus can infect from the lateral ventricle, however, it continues to divide somewhere in the parenchyma of the brain.

4. Non-pyramidal cells that are considered to be GABAergic neurons observed in the fetal lateral ventricle of the brain when an adenovirus is injected into the lateral ventricle of the brain of mouse E15 embryo are classified into subtypes with different forms. It is considered that the respective types are produced from different stem cells. When infection is carried out with an adenovirus, which does not contain an SV40 origin nor proliferate during the cell growth, substantially the same subtype of non-pyramidal cell is hardly observed in the same sample, and there is no case where the same subtype of non-pyramidal cell is observed in the vicinity thereof. On the contrary, when infection is carried out with an adenovirus, which contains an SV40 origin and proliferates as the cell proliferates during the cell growth, it was observed that the same subtype of plural non-pyramidal cells were frequently distributed close to each other. This observation result indicates that one precursor cell of GABAergic neuron produces the same subtype of non-pyramidal cell in the parenchyma of the brain.

The inventor of this application found that, while the neural stem cells present in the ventricular zone of the ganglionic eminence are GAD67 negative, precursor cells of GABAergic neurons in the cerebral cortex are GABA synthase GAD67 positive although they do not secrete GABA by forming a neural circuit as GABAergic neurons. This finding is a clear difference in characters to distinguish neural stem cells that produce many types of cells and precursor cells of GABAergic neurons that produce GABAergic neurons alone in a cell lineage of differentiation of GABAergic neuron in the human brain cortex.

With regard to an attempt of expressing GFP by using such an activity of GAD67 promoter, the case where a DNA in which a cDNA of GFP was attached to GAD67 promoter was introduced into a GABAergic neuron with a gene gun has been reported (Jin et al., Cereb Cortex 2001 11: 666-678). In addition, in a GAD67-GFP knock-in mouse in which a cDNA of GFP was attached to the downstream of GAD67 promoter, was incorporated on the chromosome, GFP was expressed in the GAD67-positive cells with almost 100% accuracy (Tamamaki et al., 2003). The precursor cells of GABAergic neurons were observed by using this mouse, and as a result, it was observed that green fluorescence was emitted (Nakamura et al., 2003). By utilizing this difference, precursor cells of GABAergic neurons and GABAergic neurons are separated from the neural stem cells or other types of cells, and further only the precursor cells of GABAergic neurons are separated. In addition, it is known that GAD67 gene and GAD65 gene coexist in almost all cells in the cerebral cortex from the fetal period (Dupuy and Houser, 1996), by using GAD65 promoter instead of GAD67 promoter, almost the same results can be obtained.

It has been shown that a precursor cell of GABAergic neuron in vivo has the activity of GAD67 promoter by the study of the applicants (Nakamura et al., 2003), and it has been reported that there is also a cell that has the activity of GAD67 promoter among the cells derived from embryo stem cells or neural stem cells (Westmoreland et al., 2001).

By attaching a reporter gene emitting fluorescence detectable even in vivo or a drug-resistance gene to the downstream of GAD67 or GAD65 promoter, and introducing it into a cell population containing a precursor cell of GABAergic neuron and a GABAergic neuron, the precursor cell of GABAergic neuron and the GABAergic neuron can be confirmed based on the fluorescence from the reporter protein or the drug resistance. The precursor cell of GABAergic neuron and the GABAergic neuron emitting fluorescence can be separated with a cell sorter, and the precursor cell of GABAergic neuron and the GABAergic neuron with drug resistance can be separated by adding a drug to a culture medium. In this case, if the cells are cultured and grown in a prolonged time in the culture medium, only the precursor cell of GABAergic neuron having division capability can be obtained.

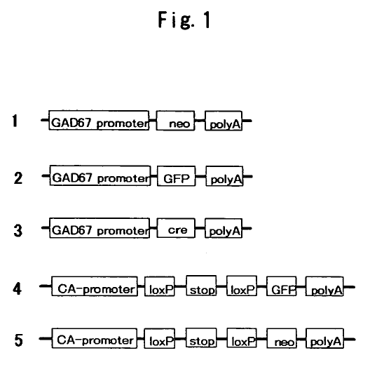

For example, if a DNA in which a cDNA of a green fluorescent protein (GFP), which is a jellyfish protein emitting green fluorescence, is attached to GAD67 promoter (DNA construct 1 in FIG. 1) is introduced by using a reagent or a virus for introducing DNA into a cell, by the electroporation method or the like, the precursor cell of GABAergic neuron and the GABAergic neuron emit green fluorescence. In the case where GAD65 promoter is used, the same effects can be obtained. The tissue containing this precursor cell of GABAergic neuron is excised, and the individual cells are dispersed by the treatment with 0.05% trypsine-EDTA, and the cell suspension is applied to a cell sorter, whereby the precursor cell of GABAergic neuron and the GABAergic neuron can be separated. With regard to the separated cells, by using a conditioned medium in which a slice of a brain containing the cerebral cortex and the ganglionic eminence have been cultured, the separated precursor cells of GABAergic neurons and the GABAergic neurons are cultured and grown, whereby the precursor cells of GABAergic neurons grow, and the GABAergic neurons reduce in number gradually.

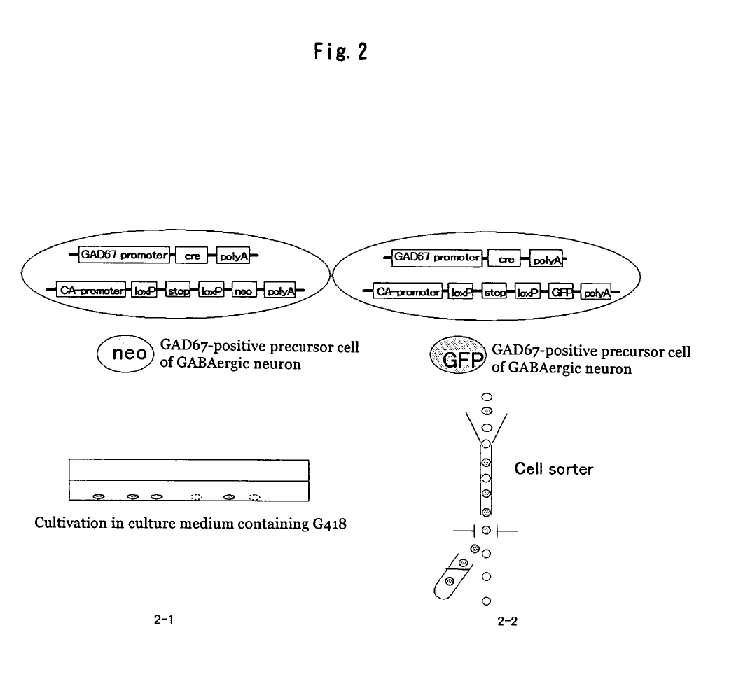

Instead of GFP, a neomycine-resistance gene is attached to GAD67 promoter or GAD65 promoter (DNA construct 2 in FIG. 2), and it is introduced into a cell group containing the precursor cell of GABAergic neuron. By cultivation is carried out by adding Geneticin (G418) to a culture medium when the cell group is subjected to dispersion culture, cells other than the precursor cell of GABAergic neuron that has the activity of GAD67 promoter die, therefore, only the precursor cell of GABAergic neuron can be selected.

However, the activity of GAD67 promoter or GAD65 promoter varies according to the cell cycle of the precursor cell of GABAergic neuron, and moreover, it is always lower than that of GABAergic neuron in general. Even if GAD67 promoter or GAD65 promoter is attached to a DNA of GFP or neomycin as it is, the intensity of the activity of GAD67 promoter or GAD65 promoter may vary depending on the primary GABAergic neuron progenitor or the secondary GABAergic neuron progenitor, which are classified in accordance with the cell lineage by us at present, therefore, it is considered that the recovery efficiency may be affected depending on the types of the precursor cells of GABAergic neurons. In order to eliminate this effect, the DNA constructs such as DNA constructs 3 to 5 in FIG. 1 are prepared. In the DNA construct 3 in FIG. 1, Cre recombinase is used as a DNA recombinant protein, however, it is not intended that the DNA recombinant protein, which can be used, is limited to Cre recombinase. In the DNA constructs 4 to 5 in FIG. 1, a reporter DNA which can visualize a cell in a living organism (e.g., GFP) or a drug-resistance gene DNA (e.g., neomycin-resistance gene) is placed between two DNA sequences (e.g., loxP), which are recognized by a DNA recombinant enzyme and are arranged in the forward direction, and it is attached to a DNA containing a promoter for ubiquitous expression (e.g., CA promoter, Japanese Patent Nos. 2824433 and 2824434) in advance. These two DNA constructs (3 and 4 or 3 and 5) are introduced into a cell group containing the precursor cell of GABAergic neuron. In the precursor cell of GABAergic neuron and the GABAergic neuron, since they have the activity of GAD67 or GAD65 promoter, a DNA recombinant enzyme is expressed, recombination occurs between th two DNA sequences recognized by the DNA recombinant enzyme, and the stop codon present therebetween is removed. As a result, in the case where a reporter that can visualize a cell in a living organism (e.g., GFP) is expressed, the precursor cell of GABAergic neuron and the GABAergic neuron can be separated by using a cell sorter, and in the case where a drug-resistance protein is expressed, only the precursor cell of GABAergic neuron and the GABAergic neuron can be selected by adding a drug (e.g., Genetisin) to a culture medium (FIG. 2). Further, cultivation is continued by using a conditioned medium in which a slice of a brain containing the cerebral cortex and the ganglionic eminence have been cultured, whereby the precursor cells of GABAergic neurons grow, and the GABAergic neurons reduce in number gradually.

Heretofore, by conditioning the culture condition for an embryo stem cell or a neural stem cell, various cells such as a GABAergic neuron have been induced. In any condition, however, there was no system that produces only a single type of cell, therefore, it was necessary to separate cells again before they were used for a treatment, which resulted in a loss of cell activity. According to the invention of this application, the precursor cells of GABAergic neurons can be obtained in high purity. The precursor cell of GABAergic neuron is GAD67 and GAD65 positive, and produces GAD67-positive and GAD65-positive cells. Therefore, considering that the GAD67-positive and GAD65-positive cells are only GABAergic neurons in the brain, by culturing the precursor cell of GABAergic neuron, a system that produces only the precursor cell of GABAergic neuron and the GABAergic neuron is provided.

Hereunder, the invention of this application will be described in more detail and specifically with reference to Example, however, the invention of this application is not limited to the following examples.

### EXAMPLE

By using a mouse, GAD67-GFP knock-in mouse, in which a DNA construct obtained by attaching a cDNA of GFP to just downstream of GAD67 promoter was inserted into the genomic DNA by using the gene targeting method and utilizing homologous recombination, precursor cells of GABAergic neurons in the cerebral cortex were separated. The state in which the DNA construct 1 in FIG. 1 was introduced into the genome was formed in all the cells of this mouse. Therefore, all the cells having the activity of GAD67 promoter express GFP. On the contrary, all the cells expressing GFP are GAD67 positive. It has been examined in advance and has been already reported that such a cell can be considered to be a precursor cell of GABAergic neuron and a GABAergic neuron (Tamamaki et al., submitted).

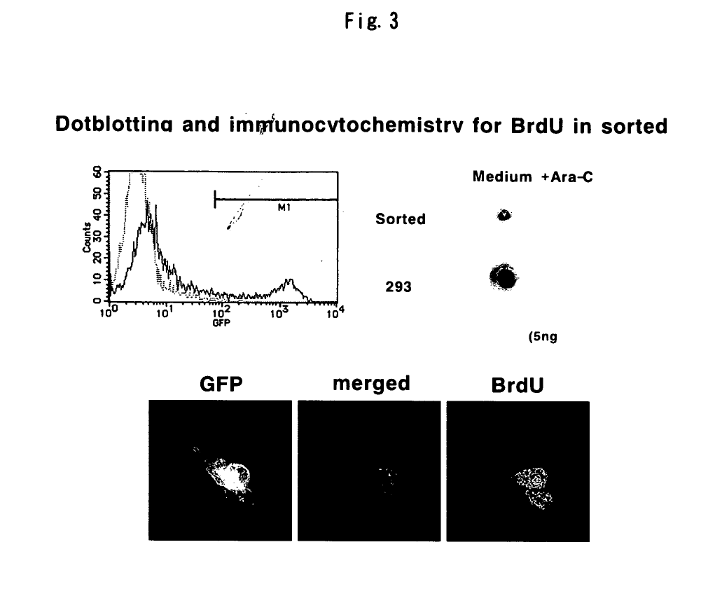

The cerebral cortex was taken out from a GAD67-GFP knock-in mouse shortly after birth or on embryonic day 18, in which the precursor cells of GABAergic neurons continue to produce GABAergic neurons in the cerebral cortex, and treated with 0.5% trypsin protease to partially digest the extracellular matrix and the cell adhesion molecule, whereby the cells were dispersed. The dispersed cells were soaked in PBS and applied to FACS (fluorescence activated cell sorter), and then the GFP-positive cells were collected in a culture medium (FIG. 2-2). The fluorescence intensity of GFP of the cells collected at this time is shown in the upper left of FIG. 3. The culture medium used for the cultivation of the collected precursor cells of GABAergic neurons and GABAergic neurons was a conditioned medium obtained by culturing the cerebral cortex and the ganglionic eminences for one day in a basal culture medium to which only a cell growth factor, which is described in the neurosphere method (Reynolds and Weiss, 1992; Vescovi et al., 1993; Gritti et al., 1996), was added and then removing the cells with a filter.

After collecting the cells, in the culture medium, part of the precursor cells of GABAergic neurons began to undergo cell division. The lower figure of FIG. 3 shows the cells having undergone cell division, and indicates that BrdU which had been mixed in the culture medium beforehand in order to confirm the cells having undergone cell division was incorporated into the DNA in the nuclear. At this time, a DNA synthase inhibitor had been added to the culture medium, BrdU was not incorporated into the DNA as shown in the upper right of FIG. 3. Therefore, it was confirmed that BrdU was incorporated by cell proliferation. In addition, both two daughter cells after division are GFP positive, and it is considered that two primary precursor cells of GABAergic neurons, or one primary precursor cell of GABAergic neuron and one secondary precursor cell of GABAergic neuron, or two secondary precursor cells of GABAergic neurons, or two GABAergic neurons were produced. In any case, it was expected that if the culture condition thereafter is conditioned, only GABAergic neurons and cells associated with GABAergic neurons continue to be produced.

### INDUSTRIAL APPLICABILITY

Heretofore, by conditioning the culture condition for an embryo stem cell or a neural stem cell, various cells such as a GABAergic neuron have been induced. In any condition, however, there was no system that produced only a single type of cell, therefore, it was necessary to separate cells again before they were used for a treatment, which resulted in a loss of cell activity. According to the invention of this application, precursor cells of GABAergic neurons can be obtained in high purity. The precursor cell of GABAergic neuron is GAD67 and GAD65 positive, and produces GAD67-positive and GAD65-positive cells. Therefore, considering that the GAD67-positive and GAD65-positive cells are only GABAergic neurons in the brain, by culturing the precursor cell of GABAergic neuron, a system that produces only the precursor cell of GABAergic neuron and the GABAergic neuron is provided.

1. A method for separating a precursor cell producing a GABAergic neuron alone, which comprises the steps of:
(a) preparing a cell population containing a precursor cell of GABAergic neuron;
(b) introducing a DNA, in which a cDNA of a reporter protein emitting a signal detectable even in vivo is attached to the downstream of a promoter of GAD67 gene or GAD65 gene that is gene of an inhibitory neurotransmitter GABA synthase, into each cell in the cell population;
(c) isolating a GABAergic neuron and a precursor cell of GABAergic neuron based on the presence/ absence of the signal emitted by the reporter; and
(d) isolating the precursor cell of GABAergic neuron based on the possession of proliferative capability.

2. A method for separating a precursor cell producing a GABAergic neuron alone, which comprises the steps of:
(a) preparing a cell population containing a precursor cell of GABAergic neuron;
(b) introducing a DNA, in which a cDNA of a protein imparting a property of drug resistance is attached to the downstream of a promoter of GAD67 gene or GAD65 gene that is gene of an inhibitory neurotransmitter GABA synthase, into each cell in the cell population;
(c) isolating a GABAergic neuron and a precursor cell of GABAergic neuron based on the presence/absence of the drug resistance; and
(d) isolating the precursor cell of GABAergic neuron based on the possession of proliferative capability.

3. A method for separating a precursor cell producing a GABAergic neuron alone, which comprises the steps of:
(a) preparing a cell population containing a precursor cell of GABAergic neuron;
(b) introducing a DNA, in which a cDNA of a recombinant enzyme and a cassette DNA are attached to the downstream of a promoter of GAD67 gene or GAD65 gene that is a gene of an inhibitory neurotransmitter GABA synthase, into each cell in the cell population, wherein the cassette DNA expresses a reporter protein emitting a signal detectable even in vivo after being genetically recombined;
(c) isolating a GABAergic neuron and a precursor cell of GABAergic neuron based on the presence/absence of the signal emitted by the reporter; and
(d) isolating the precursor cell of GABAergic neuron based on the possession of proliferative capability.

4. A method for separating a precursor cell producing a GABAergic neuron alone, which comprises the steps of:
(a) preparing a cell population containing a precursor cell of GABAergic neuron;
(b) introducing a DNA, in which a cDNA of a recombinant enzyme and a cassette DNA are attached to the downstream of a promoter of GAD67 gene or GAD65 gene that is gene of an inhibitory neurotransmitter GABA synthase, into each cell in the cell population, wherein the cassette DNA expresses a protein imparting a property of drug resistance after being genetically recombined;
(c) isolating a GABAergic neuron and a precursor cell of GABAergic neuron based on the presence/absence of the drug resistance; and
(d) isolating the precursor cell of GABAergic neuron based on the possession of proliferative capability.

5. The method according to any one of claims 1 to 4, wherein in the step (a), the cell population contains a precursor cell of GABAergic neuron induced from an embryo stem cell or a neural stem cell.

6. The method according to any one of claims 1 to 4, wherein in the step (a), the cell population is prepared by dispersing tissues containing a precursor cell of GABAergic neuron of a donor.

7. The method according to any one of claims 1 to 4, wherein the method of introducing the DNA in the step (b) includes transformation mediated by a virus.

8. The method according to any one of claims 1 to 4, wherein the method of introducing the DNA in the step (b) includes electroporation.

9. The method according to any one of claims 1 to 4, wherein the method of introducing the DNA in the step (b) includes transformation mediated by a liposome.

10. The method according to claim 5, wherein the embryo stem cell or the neural stem cell is derived from a mammal.

11. The method according to claim 10, wherein the mammal is human.

12. The method according to claim 6, wherein the donor is a mammal.

13. The method according to claim 12, wherein the mammal is human.

14. The method according to any one of claims 1 to 4, further comprising a step of transplanting the cell separated in the step (d) into a recipient.

15. A precursor cell producing a GABAergic neuron alone, which is obtained by the method according to any one of claims 1 to 4.

16. A kit containing a reagent and a cell, which is used for obtaining a precursor cell producing a GABAergic neuron alone in a method according to any one of claims 1 to 4.
